(19) United States
(12) Patent Application Publication
(10) Pub. No.: US 2022/0042107 A1
(43) Pub. Date: Feb. 10, 2022

(54) SYSTEMS AND METHODS OF SCORING RISK AND RESIDUAL DISEASE FROM PASSENGER MUTATIONS

(71) Applicant: Agendia NV, Amsterdam (NL)

(72) Inventors: Bastiaan Van Der Baan, Irvine, CA (US); Annuska Maria Glas, Irvine, CA (US)

(21) Appl. No.: 17/395,013

(22) Filed: Aug. 5, 2021

Related U.S. Application Data
(60) Provisional application No. 63/062,115, filed on Aug. 6, 2020.

(51) Int. Cl. C12Q 1/6886

(52) U.S. Cl. CPC C12Q 1/6886; C12Q 2600/156

(57) ABSTRACT

In particular, systems and methods of the invention measure amounts of passenger mutations, and optionally driver mutations, to predict risk of cancer-related pathogenicity. Preferably, the mutations are measured from sequences reads. Sequence reads of patient-derived nucleic acids may be compared with one or more references to identify and measure the amount of passenger mutations and optionally driver mutations. The amount of passenger and driver mutations may be correlated to known associations with recurrent and residual disease to predict a risk of cancer recurrence.

### TECHNICAL FIELD

This invention relates to oncology. In particular, this invention provides systems and methods for predicting a risk of recurrent or residual disease by assessing passenger mutations.

### BACKGROUND

Genomic instability is a cornerstone of cancer. This is because genomic instability gives rise to mutations that allow cells to acquire new traits, which ultimately lead to uncontrolled growth. These new traits are acquired by “driver” mutations in cancer genes (i.e., oncogenes). Most mutations, however, are unimportant for cancer growth and these are termed “passenger” mutations. The role that passenger mutations play in cancer and other adaptive processes is presently unknown.

Driver mutations are a primary focus of therapeutic research as driver mutations are casually connected to cancer and offer clinically actionable targets. For example, clinicians may develop and use therapeutics that target driver mutations to specifically eliminate cells carrying them with the intention of curing the cancer.

Unfortunately, many patients develop recurrent disease following treatment, suggesting not all of the cancer cells were eliminated. Assays for residual disease based on the driver mutations are likely to return false positive results, as the cells carrying those mutations were eliminated by the treatment.

### SUMMARY

This disclosure relates to methods for discovering residual disease by expanding screens to include passenger mutations. Since therapeutics often eliminated cells with driver mutations, screening for passenger mutations increases sensitivity for residual disease. Moreover, passenger mutations are correlated with genetic instability. The more unstable the genome, the higher number of passenger mutations that are likely arise. Accordingly, methods of the invention involve screening for markers of genomic instability, which is a signature trait of cancer cells. Screening for passenger mutations, and optionally driver mutations, allow for the early detection of residual disease. Early detection allows clinicians to quickly administer new treatments before the cancer spreads.

In particular, systems and methods of the invention measure amounts of passenger mutations, and optionally driver mutations, to predict risk of cancer-related pathogenicity. Preferably, the mutations are measured from sequences reads. Sequence reads of patient-derived nucleic acids may be compared with one or more references to identify and measure the amount of passenger mutations and optionally driver mutations. The amount of passenger and driver mutations may be correlated to known associations with recurrent and residual disease to predict a risk of cancer recurrence. Methods may further incorporate additional data sources such as patient-specific gene expression profiles and/or analyses of stained tissue images to support and/or confirm disease assessments. The output of the systems and methods described herein may include a quantitative score evaluating the pathogenic risk of recurrence or residual disease.

In one aspect, this disclosure provides a method for predicting risk of recurrent or residual cancer. The method includes the steps of measuring an amount of one or more passenger mutations from nucleic acids; correlating said amount of passenger mutations with known associations with residual or recurrent disease; and predicting risk of recurrent or residual disease based upon said correlating step. Methods may further include assessing disease severity based on the correlating step by, for example, determining a stage of cancer progression or cancer subtype. Preferably, methods further include measuring one or more driver mutations from the nucleic acids and including the measured driver mutations in the correlating step.

Preferably, the nucleic acids comprise nucleic acids that are released from cells into the patient's blood stream, e.g., cell free nucleic acids. Cell free nucleic acids may be taken from the patient by a blood draw or liquid biopsy. Analyzing the cell free nucleic acids from blood, as opposed to nucleic acids taken from solid tissue, is beneficial since obtaining the nucleic acids does not require an invasive procedure, such as a tissue biopsy, which is often painful. Preferably, the cell free nucleic acids comprise cell free tumor DNA.

One insight of the invention is that cell free nucleic acids are surprisingly stable in blood when encapsulated inside extracellular vesicles where they are protected from degradation. Accordingly, systems and methods of the invention may involve the isolation of patient cell free nucleic acids from extracellular vesicles, e.g., exosomes.

Measuring an amount of passenger mutations preferably involves sequencing. Sequencing may be performed by a variety of methods including by next generation sequencing, or third generation sequencing methods. The nucleic acids may be isolated from a patient and sequenced directly, or alternatively, the nucleic acids may be amplified by, for example, PCR based methods prior to sequencing. Sequencing the cell free nucleic acids produces a plurality of sequence reads. The sequence reads may be analyzed to detect one or more passenger mutations, and optionally, one or more driver mutations. Passenger mutations may be identified as mutations that do not show a significant statistical correlation with cancer.

Methods of the invention may involve multiple patient assays to assess changes in a patient's health over time. For example, a patient may have a first patient assay performed before or after a cancer diagnosis in which one or more passenger mutations, and optionally driver mutations, are identified and recorded. Then, at a later time, the patient may have a second patient assay performed, and the results of the second patient assay may be correlated with the first patient assay to measure changes in the amount and/or pattern of passenger and driver mutations. Changes in the amount and/or pattern of the mutations may inform on the patient's health. For example, the appearance of one or more passenger mutations, and optionally driver mutations, above a statistically significant deviation may indicate recurrence of disease.

Methods of the invention may involve the step of analyzing a plurality of sequence reads, wherein analyzing comprises detecting one or more passenger mutations previously identified in a first patient assay. The first patient assay may have been performed at least one month before performing the steps of the method. In some embodiments, the steps of the method are performed after the patient received a treatment and the first patient assay was performed before said treatment. Accordingly, methods of the invention may be useful to determine whether the treatment was effective.

This disclosure also provides methods of sample preparation to screen for passenger mutations. Methods may include targeted enrichment technologies. For example, methods of the invention may include the targeted enrichment of a target nucleic acid from a sample of nucleic acids wherein the target nucleic acids is suspected of having one or more passenger mutations that are informative of disease. Targeted enrichment may be performed with one or more hybridization probes. The hybridization probes may be designed to be complementary to regions identified as having passenger mutations in a first patient assay, or suspected of having mutations on account of clinical data obtained by evaluating numerous patients over time. The hybridization probes may be added to a library of nucleic acids and used to enrich for the target nucleic acids suspected of harboring the mutations. Accordingly, in some embodiments, methods of the invention involve enriching for chromosomal regions suspected of having at least one passenger mutation previously identified in a first patient assay and measuring the one or more passenger mutations in those chromosomal regions. The enriching may comprise using hybridization probes to capture and isolate chromosomal regions having at least one passenger mutation.

Methods may incorporate additional data sources such as, for example, patient-specific gene expression profiles and/or analyses of stained tissue images to support and/or confirm disease assessments. Data collected from such analyses may be useful to support or confirm assessments made by measuring amounts of mutations. Thus, systems and methods of the invention may further include the step of determining an expression level for one or more gene transcripts and including the determined expression levels in the predicting step. The one or more gene transcripts may be genes associated with cancer. For example, in some embodiments, methods of the invention may include harvesting cell free nucleic acids from a patient, wherein the cell free nucleic acids comprise RNA. The RNA may be converted into complementary DNA and sequenced.

### BRIEF DESCRIPTION OF THE DRAWINGS

FIG. 1 shows a method for predicting risk of recurrent or residual cancer.

FIG. 2 shows a sample comprising nucleic acids.

FIG. 3 shows a report of tumor-related mutations for a patient.

### DETAILED DESCRIPTION

Cancers arise from an accumulation of mutations. Mutations include changes to DNA sequences, e.g., insertions, deletions, and single nucleotide polymorphisms, and may be categorized as either driver mutations or passenger mutations. Driver mutations confer growth advantage on the cells carrying them and have been positively selected during the evolution of the cancer. They reside, by definition, in a subset of genes known as cancer genes or oncogenes. Passenger mutations are generally not selected for, and do not conferred a clonal growth advantage. Passenger mutations are generally not associated with cancer development. Passenger mutations may be found within cancer genomes because somatic mutations without functional consequences often occur during cell division. Thus, a cell that acquires a driver mutation will may already have biologically inert somatic mutations within its genome. It is an insight of the invention that these may be carried along in the clonal expansion that follows and therefore may present in all cells of the final cancer, as well as the cells that preceded the cancer, making the passenger mutations a useful marker for recurrent and residual disease.

One challenge of treating cancer is that survival of a single tumor cell may be sufficient to cause recurrence. Cancer therapies are designed to kill or eliminate 100% of cancer cells, but even treatment methods that result in resolution of all clinical signs of the disease may still leave a handful of residual cancer cells in the patient's body. Because their proliferation is unregulated, the remaining cancer cells may expand in number and invade other tissues. When cancer cells are few in number and have not metastasized, their presence is difficult to detect. Thus, for a cancer patient who has already achieved a defined benchmark from a course of therapy, it is often difficult to determine whether or when additional treatment is necessary. The invention addresses the foregoing problem by providing methods for detecting residual cancer cells in a patient.

Systems and methods of the invention rely on measured amounts of passenger mutations, and optionally driver mutations, to predict risk of cancer-related pathogenicity. Preferably, the mutations are measured from sequences reads. As discussed below, sequence reads of patient-derived nucleic acids may be compared with one or more references to identify and measure the amount of passenger mutations and optionally driver mutations. The one or more references may include sequences from one or more other subjects with and without cancer. The references may be annotated to identify known tumor-associated mutations and thus provide a successful match, or “hit” on any sequence from patient-specific nucleic acids to which the sequence read is mapped. Alternatively, or in addition to, the reference may comprise sequences from the patient's own healthy genome. For example, the reference may comprise sequences of DNA either collected from the patient prior to a cancer diagnosis, or taken from a healthy tissue.

The amount of passenger and driver mutations may be correlated to known associations with recurrent and residual disease. For example, the presence of a number of passenger mutations above a statistically significant threshold may be indicative of genome instability, and thus, reflect a high likelihood of cancer recurrence.

Methods of the invention may include tracking one or more driver mutations. In some instances, the driver mutations may arise from a passenger mutation. One important subclass of driver mutation is a mutation that confers resistance to cancer therapy. These are typically found in recurrences of cancers that have initially responded to treatment but that are now resistant. Resistance mutations often confer limited growth advantage on the cancer cell in the absence of therapy. Some seem to predate initiation of treatment, existing as passengers in minor subclones of the cancer cell population until the selective environment is changed by the initiation of therapy. The passenger is then converted into a driver and the resistant subclone preferentially expands, manifesting as the recurrence.

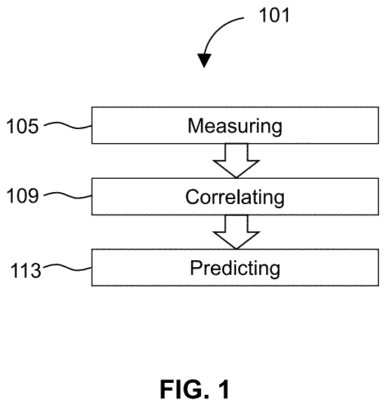

FIG. 1 shows a method 101 for predicting risk of recurrent or residual cancer. The method includes measuring 105 an amount of one or more passenger mutations from nucleic acids; correlating 109 said amount of passenger mutations with known associations with residual or recurrent disease; and predicting 113 risk of recurrent or residual disease based upon said correlating step.

The method 101 may further include obtaining a sample comprising nucleic acids from a patient prior to the measuring 105 step. The sample may be a body fluid sample or a tissue sample. A body fluid sample may comprise one of blood, saliva, sputum, urine, semen, transvaginal fluid, cerebrospinal fluid, sweat, or stool. A tissue sample may comprise soft or hard tissue. The sample may be processed to isolate patient nucleic acids. The nucleic acids may comprise DNA or RNA or a combination thereof.

In some instances, the nucleic acids comprise cell free nucleic acids taken from body fluid. Making assessments from cell free nucleic acids taken from a body fluid offers important advantages over those taken from solid tissue. For example, cell free nucleic acids may be taken from body fluid, such as blood, by routine blood draws, which is substantially pain free. Whereas, solid tissue requires invasive and painful biopsy procedures in order to obtain genetic material. Moreover, analysis of cell free nucleic acids, e.g., circulating tumor DNA, allows a researcher or clinician to detect and analyze tumor DNA before the tumor is visible. Conversely, obtaining a tissue sample comprising tumor DNA generally requires that the tissue present symptoms of tumor in order to identify the tissue to be removed.

In preferred embodiments, the sample comprises blood, as it is an insight of the invention that cell free nucleic acids are surprisingly stable in blood when encapsulated inside extracellular vesicles where they are protected from degradation. Accordingly, the method 101 may further involve segregating extracellular vesicles from a patient blood sample and subsequently isolating cell free nucleic acids from the segregated vesicles prior to the measuring 105 step, as described below. Preferably, the body fluid sample is taken from a patient that has been recently treated for cancer. The cancer may be one of bladder cancer; breast cancer; colorectal cancer; kidney cancer; lung cancer; lymphoma; skin cancer; oral cancer; pancreatic cancer; prostate cancer; thyroid cancer; or uterine cancer. Preferably the cancer comprises breast cancer.

Measuring 105 an amount of one or more passenger mutations, and optionally driver mutations, preferably involves sequencing the nucleic acids to reveal the mutations. Sequencing may be performed by a number of methods known in the art, for example, by next generation sequencing methods or third generation sequencing. For example, see, generally, Quail, et al., 2012, A tale of three next generation sequencing platforms: comparison of Ion Torrent, Pacific Biosciences and Illumina MiSeq sequencers, BMC Genomics 13:341. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. An example of a sequencing technology that can be used is Illumina sequencing. Illumina sequencing is based on the amplification of DNA on a solid surface using fold-back PCR and anchored primers. Genomic DNA is fragmented and attached to the surface of flow cell channels. Four fluorophore-labeled, reversibly terminating nucleotides are used to perform sequential sequencing. After nucleotide incorporation, a laser is used to excite the fluorophores, and an image is captured and the identity of the first base is recorded. Sequencing according to this technology is described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

Once the nucleic acids are sequenced, the sequence reads may be analyzed to identify mutations. Standard, widely used protocols for analysis usually involve comparing each sequence to a human reference genome. Methods of the invention may involve aligning sequences reads obtained from a subject of interest to one or more reference genomes, for example, GRC37, and then using computational methods to identify high-confidence differences, including single-nucleotide polymorphisms (SNPs), small insertions and deletions (indels), and copy-number variants. However, because of natural variation in the human population, comparing patient obtained sequences to GRC37 may yield a high number of differences making it difficult to assess which mutations are patient specific, and as such, it may be difficult to identify mutations with prognostic value. Accordingly, in some instances, methods may involve aligning the patient obtained sequences to sequences obtained from healthy tissue taken from the patient.

The sequence reads from patient nucleic acids may be aligned to a patient-specific genomic reference graph. Since the patient-specific genomic reference graph may include non-tumor sequences from the patient, any difference between the reads from the patient nucleic acids and the patient-specific genomic reference graph is presumptively a tumor-associated mutation, e.g., a passenger or driver mutation.

Mutations identified by alignment to the patient specific genomic reference graph may include both driver mutations and passenger mutations. Measuring 105 the one or more passenger mutations will therefore require distinguishing among these two mutation types. One insight of the invention is the recognition that because driver mutations confer growth advantage on the cancer cells carrying them, driver mutations, by definition, will reside in a subset of genes known as cancer genes or oncogenes. Thus, in some embodiments, driver mutations may be identified as the mutations that overlap, or partially overlap, with an oncogene. Identifying sequences that correspond to oncogenes may be done by analyzing recurrence data from prior large-scale human breast sequencing projects, for example, The Cancer Genome Atlas (TCGA).

Measuring 105 the one or more passenger mutations may be performed by aligning sequence reads from patient nucleic acids and a patient-specific genomic reference graph; identifying all the differences; and annotating each difference as either a driver mutation or passenger mutation. Annotating driver and passenger mutations may depend on whether the mutation occurs within a sequence associated with an oncogene. Mutations that do not occur within a sequence corresponding to an oncogene may be annotated as passenger mutations. The total number of passenger mutations, and optionally driver mutations, may be quantified by counting. Preferably, the counts are normalized for total number of reads aligning at that a position. The normalization step is recommended to correct for variation in coverage.

It may be desirable to, at a later point in time (e.g., after a treatment) to align a new set of reads from a newly-sequenced patient sample. The new set of reads may be aligned with, for example, sequences of patient nucleic acids comprising one or more mutations. Then, systems of the invention may be used to report on changes in the patient's mutational profile over a course of time.

Preferably, the alignment step and identifying step are performed by one or more processors of a computer system wherein the one or more genomic reference graphs are stored within a non-transitory, tangible memory subsystem.

After measuring 105 one or more passenger mutations from patient-derived nucleic acids, the amount of passenger mutations measured 105 are correlated 109 with known associations or cancer recurrence and residual disease. Without being bound to any one hypothesis, methods of the invention recognize that an accumulation of passenger mutations are an inextricable consequence of genomic instability. As such, a measured 105 increase in the amount of passenger mutations is reflective of an increase in genomic instability. And the more unstable the genome, the more likely a cell is to acquire a driver mutation that leads to the development of a cancer. Thus, it is an insight of the invention that a measured 105 increase in passenger mutations, for example, an increase of about 1%, 5%, 10%, 15%, 20%, or greater, is correlated 109 to an increased risk of residual or recurrent disease.

Correlating 109 is preferably performed with a computer system. The computer system preferably hosts a machine learning system. The machine learning system may be trained to identify correlations between passenger mutation patterns, and optionally driver mutations, with patterns of patients having known outcomes and based on the correlation, predict a risk of recurrence or residual disease.

After correlating 109 the amount of passenger mutations with known associations, the correlation may be used to predict 113 a risk of recurrent or residual disease. For example, an increase in the amount of passenger mutations above, for example, 10%, may indicate a moderate risk of recurrent or residual disease. An increase in the amount of passenger mutations above, for example, 15%, may indicate a high risk of residual disease.

The method 101 may further include obtaining a sample comprising nucleic acids from a patient prior to the measuring 105 step.

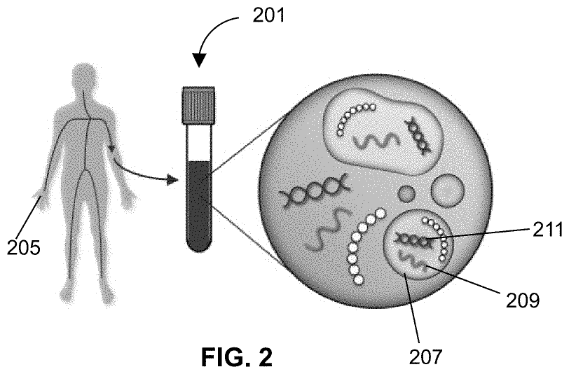

FIG. 2 shows a sample 201 comprising nucleic acids. The sample 201 comprises blood 203 that is preferably taken from a patient 205 by blood draw. The blood 203 contains extracellular vesicles 207, which are small plasma membrane-encapsulated particles released from all cells that can enter into the bloodstream. Extracellular vesicles 207 comprise exosomes and microvesicles. Exosomes are small extracellular vesicles (50-100 nanometers) of endocytic origin while microvesicles are larger particles (100-1,000 nanometers) that are shed via direct cell membrane budding.

Extracellular vesicles 207 contain proteins (tumor antigens, immunosuppressive, and/or angiogenic molecules) and nucleic acids in the form of cell free nucleic acids, including cell free RNA 209 and cell free DNA. In some instances, the cargo of extracellular vesicles 207 may be analyzed to determine their cell of origin by, for example, by segregating the extracellular vesicles 207 and sequencing the nucleic acids contained therein or performing an immunochemistry staining for cell-type specific proteins. In some cases, the extracellular vesicles 207 may be segregated by immunostaining the extracellular vesicles 207 for a protein that is over or under expressed in cancer, and subsequently sorting the stained extracellular vesicles 207 by FACS. Accordingly, methods of the invention may include the step of determining an extracellular vesicle's origin (e.g., determining that the vesicle was released from a tumor cell) based on the content of the extracellular vesicle before identifying at least two of the cell free nucleic acids contained therein, as described below.

By determining the extracellular vesicle's origin prior to identifying the cell free nucleic acids, a research or clinician, may focus their analyses specifically on nucleic acids associated with tumor cells. Extracellular vesicles 207 are ubiquitous in body fluids including plasma, cerebral spinal fluid, aqueous humor, amniotic fluid, saliva, synovial fluid, adipose tissue, and urine. Both plasma and cerebral spinal fluid extracellular vesicles including exosomes are a useful source of cell free nucleic acids for assessing disease. Accordingly, methods of the invention allow for the analyses of extracellular vesicles cargo, to track and predict tumor growth and allow early treatment for patients. Alternatively, patients with treatment-related pseudo-progression may be spared unnecessary and potentially ineffective changes in treatment strategy.

In some embodiments, the sample is collected by blood draw or by fine needle aspiration and the cell free nucleic acids are extracted from extracellular vesicles, such as exosomes, present in the blood sample. Isolating the extracellular vesicles from the body fluid sample may be required. To isolate extracellular vesicles from the body fluid sample a method of differential ultracentrifugation (low-speed centrifugation to remove cells and debris, high-speed ultracentrifugation to pellet exosomes) may be performed. For example, to isolate extracellular vesicles from blood the sample, the sample may be centrifuged at low speeds allowing for the removal of cells and debris by, for example, pipetting or dumping out supernatant. The sample may then be centrifuged at high speeds, for example, at 100,000×g for 70 min, to pellet the extracellular vesicles allowing the extracellular vesicles to be separated from remaining material. Easy-to-use precipitation solutions, such as the precipitation solution sold under the trade name ExoQuick by System Biosciences, may be used to precipitate the vesicles in liquid. Once the vesicles are isolated, the vesicles may be lysed in lysis buffer to release the cell free nucleic acids. For example, as described Garcia, 2019, Isolation and Analysis of Plasma-Derived Exosomes in Patients With Glioma, Front Oncol, 9: 651, incorporated by reference.

In preferred embodiments, the nucleic acids comprise cell free nucleic acids, preferably cell free DNA. Any suitable method may be used to isolate cell free DNA and it may be preferable to use a commercially-available kit such as the circulating nucleic acid kit sold under the trademark QIAAMP by Qiagen (Venlo, Netherlands) or the plasma/serum cell free circulating DNA purification mini kit sold by Norgen Biotek Corp. (Ontario, Canada). After isolation, the sample includes DNA in a form amenable to sequencing, e.g., by next-generation sequencing (NGS) instruments. In some instances, the nucleic acids comprise cell free RNA (cfRNA), which may include messenger RNA (mRNA), microRNA (miRNA), long non-coding RNA (lncRNA), and circular RNA (circRNA). The cell free RNA may or may not be fragmented to a desired size. Fragmenting may be performed using sonication methods or by enzyme treatment. Preferably, the isolated cfRNA comprises a 260/280 and 260/230 absorbance ratio values of close to 2.0.

Methods of the invention may involve multiple patient assays, i.e., recitation of the steps of FIG. 1, to assess changes in a patient's health over time. For example, a patient may have a first patient assay performed before or after a cancer diagnosis in which one or more passenger mutations, and optionally driver mutations, are identified. The identified mutations may be stored, for example, on a hard drive of a computer. Then, at a later time, the patient may have a second patient assay performed, and the results of the second patient assay may be correlated with the first patient assay to measure changes in the amount and/or pattern of passenger and driver mutations. Changes in the amount and/or pattern of the mutations may be used to predict the patient's health. For example, the appearance of one or more passenger mutations, and optionally driver mutations, above a statistically significant deviation may indicate a high risk of recurrence of disease.

Methods of the invention may involve the step of analyzing a plurality of sequence reads, wherein analyzing comprises detecting one or more passenger mutations previously identified in a first patient assay. The first patient assay may have been performed at least one month before performing the steps of the method. In some embodiments, the steps of the method are performed after the patient received a treatment and the first patient assay was performed before said treatment. Accordingly, methods of the invention may be useful to determine whether the treatment was effective. For example, an increase in a number of identified passenger mutations and optionally the detection of one or more new driver mutations may indicate the treatment was ineffective.

Methods of the invention may report on the patient's tumor-related mutation population and optionally provide a predictor recurrence or residual disease.

Knowledge of a mutational landscape of patient-derived nucleic acids may be used to inform on a likelihood of cancer recurrence, detect remissions, manage treatment decisions, monitor therapy, or combinations thereof. For example, where the report includes a description of a plurality of mutations, the report may also include an estimate of a tumor mutation burden (TMB) for a tumor. It may be found that TMB is predictive of success of immunotherapy in treating a tumor, and thus methods described herein may be used for treating a tumor. Methods of the invention thus may be used to detect and report clinically actionable information about a patient or a tumor in a patient. For example, the method 101 may be used to provide a report describing the presence of the genomic alteration in a genome of a subject.

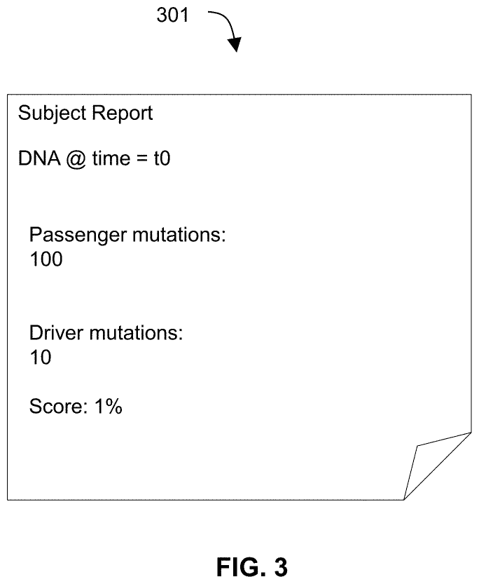

FIG. 3 shows a report 301 of tumor-related mutations for a patient. The report 301 preferably includes all known tumor-related mutations (if any), such as passenger and driver mutations, that sequence reads from patient derived nucleic acids are aligned to. The report may show what proportion of reads are aligned to each mutation type. The report may show a predictive value or index reflecting a likelihood of recurrence or residual disease based on the patient's tumor mutation profile.

This disclosure also provides methods of sample preparation to screen for passenger mutations. Methods may include targeted enrichment technologies. For example, methods of the invention may include the targeted enrichment of a target nucleic acid from a sample of nucleic acids wherein the target nucleic acids is suspected of having one or more passenger mutations that are informative of disease. The target nucleic acids may be suspected of having one or more passenger mutations on account of a previous assay, or based on clinical knowledge from having analyzed numerous patients. For example, information gleaned from having analyzed sequence data from other patients may reveal chromosomal regions that are “hot spots” for passenger mutations. Such analyses may also reveal certain patterns of passenger mutations that are indicative of recurrence or residual disease. For example, it may be found that an amount of passenger mutations positioned within, or adjacent to, genes coding for DNA repair enzymes are highly correlated with recurrence. Enriching for these regions allows a clinical or research to reduce sequencing costs.

Targeted enrichment may be performed with one or more hybridization probes. The hybridization probes may be designed to be complementary to regions identified as having passenger mutations in a first patient assay, or suspected of having mutations on account of clinical data obtained by evaluating numerous patients over time. The hybridization probes may be added to a library of nucleic acids and used to enrich for the target nucleic acids suspected of harboring the mutations. Accordingly, in some embodiments, methods of the invention involve enriching for chromosomal regions suspected of having at least one passenger mutation previously identified in a first patient assay and measuring the one or more passenger mutations in those chromosomal regions. The enriching may comprise using hybridization probes to capture and isolate chromosomal regions having at least one passenger mutation.

Methods may incorporate additional data sources such as, for example, patient-specific gene expression profiles and/or analyses of stained tissue images to support and/or confirm disease assessments. Data collected from such analyses may be useful to support or confirm assessments made by measuring amounts of mutations. Thus, systems and methods of the invention may further include the step of determining an expression level for one or more gene transcripts and including the determined expression levels in the predicting step. The one or more gene transcripts may be genes associated with cancer. For example, in some embodiments, methods of the invention may include harvesting cell free nucleic acids from a patient, wherein the cell free nucleic acids comprise RNA. The RNA may be converted into complementary DNA and sequenced.

For example, RNA may be isolated from the patient and quantified to determine levels of distinct RNA species. The RNA may be quantified, by any of a wide variety of methods, including, but not limited to, sequencing (e.g., RNA-seq), hybridization analysis, amplification e.g., via the polymerase chain reaction, for example, by reverse transcription polymerase chain reaction (RT-PCR). In preferred embodiments, quantifying the RNA involves targeted enrichment next-generation sequencing technologies, which are useful to identify specific nucleic acids of interest that preferably include at least a portion of MammaPrint and BluePrint genes. For example, as described in Mittempergher, 2019, MammaPrint and BluePrint Molecular Diagnostics Using Targeted RNA Next-Generation Sequencing Technology, The Journal of Molecular Diagnostics, Volume 21, Issue 5, 808-823, which is incorporated by reference.

Once the levels of RNA expression are determined, the levels of RNA expression may be analyzed to help predict disease outcome in breast cancer patients, for example, see, van′t Veer, 2002, Gene Expression Profiling Predicts Clinical Outcome of Breast Cancer, Nature, Vol 415, pages 530-535, incorporated by reference. The results of such analyses may be combined with assessments of passenger, and optionally driver mutations, to support or confirm predictions of recurrence or residual disease made from assaying amounts of passenger, and optionally driver, mutations.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for predicting risk of recurrent or residual cancer, the method comprising the steps of:
measuring an amount of one or more passenger mutations from nucleic acids;
correlating said amount of passenger mutations with known associations with residual or recurrent disease; and
predicting risk of recurrent or residual disease based upon said correlating step.

2. The method of claim 1, further comprising measuring one or more driver mutations from the nucleic acids and including the measured driver mutations in the correlating step.

3. The method of claim 1, wherein the nucleic acids are isolated from a blood sample from a patient.

4. The method of claim 1, further comprising the step of determining an expression level for one or more gene transcripts and including the determined expression levels in the predicting step.

5. The method of claim 1, further comprising the step of assessing disease severity based on the correlating step.

6. The method of claim 3, wherein the nucleic acids comprise cell free nucleic acids.

7. The method of claim 6, wherein the cell free nucleic acids comprise ctDNA.

8. The method of claim 7, wherein said measuring step comprises sequencing the cell free nucleic acid to produce a plurality of sequence reads.

9. The method of claim 8, further comprising the step of analyzing the plurality of sequence reads, wherein analyzing comprises detecting one or more passenger mutations previously identified in a first patient assay.

10. The method of claim 9, wherein the first patient assay was performed at least one month before performing the steps of the method.

11. The method of claim 10, wherein the steps of the method are performed after the patient received a treatment and the first patient assay was performed before said treatment.

12. The method of claim 8, further comprising identifying and recording locations of the one or more passenger mutations from the sequence reads and storing the locations in a data file for use in a future patient assay.

13. The method of claim 1, wherein the nucleic acids comprise RNA.

14. The method of claim 1, further comprising the step of enriching for chromosomal regions having at least one passenger mutation previously identified in a first patient assay and measuring one or more passenger mutations in those chromosomal regions.

15. The method of claim 14, wherein enriching comprises using hybridization probes to capture and isolate chromosomal regions having at least one passenger mutation.

16. The method of claim 1, wherein the passenger mutations comprise mutations that do not show a significant statistical correlation with breast cancer.
